(19) United States
(12) Patent Application Publication
(10) Pub. No.: US 2012/0095539 A1
(43) Pub. Date: Apr. 19, 2012

(54) Delivery Catheter Systems and Methods

(76) Inventors: Alexander Khairkhahan, Palo Alto, CA (US); Alan Klenk, San Jose, CA (US)

(21) Appl. No.: 13/272,074

(22) Filed: Oct. 12, 2011

Related U.S. Application Data
(60) Provisional application No. 61/392,881, filed on Oct. 13, 2010.

(51) Int. Cl. A61N 1/05; A61N 1/04

(52) U.S. Cl. 607/116; 607/115

(57) ABSTRACT

A leadless cardiac pacemaker comprises a housing, a plurality of electrodes coupled to an outer surface of the housing, and a pulse delivery system hermetically contained within the housing and electrically coupled to the electrode plurality, the pulse delivery system configured for sourcing energy internal to the housing, generating and delivering electrical pulses to the electrode plurality. Systems and methods for delivering the leadless cardiac pacemaker with delivery catheters are also provided. In some embodiments, the delivery catheters include first and second coaxial shafts configured to apply rotational torque to the pacemaker. In other embodiments, the pacemaker is held in place on the catheter with a tether.

### CROSS REFERENCE TO RELATED APPLICATIONS

This application claims the benefit under 35 U.S.C. 119 of U.S. Provisional Patent Application No. 61/392,881, filed Oct. 13, 2010, titled “Delivery and Retrieval Catheter Systems and Methods”, which is incorporated herein by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD

The present disclosure relates to leadless cardiac pacemakers, and more particularly, to features and methods by which they are affixed within the heart. More specifically, the present disclosure relates to features and methods for delivering a leadless cardiac pacemaker to tissue.

### BACKGROUND

Cardiac pacing by an artificial pacemaker provides an electrical stimulation of the heart when its own natural pacemaker and/or conduction system fails to provide synchronized atrial and ventricular contractions at rates and intervals sufficient for a patient's health. Such antibradycardial pacing provides relief from symptoms and even life support for hundreds of thousands of patients. Cardiac pacing may also provide electrical overdrive stimulation to suppress or convert tachyarrhythmias, again supplying relief from symptoms and preventing or terminating arrhythmias that could lead to sudden cardiac death.

Cardiac pacing by currently available or conventional pacemakers is usually performed by a pulse generator implanted subcutaneously or sub-muscularly in or near a patient's pectoral region. Pulse generator parameters are usually interrogated and modified by a programming device outside the body, via a loosely-coupled transformer with one inductance within the body and another outside, or via electromagnetic radiation with one antenna within the body and another outside. The generator usually connects to the proximal end of one or more implanted leads, the distal end of which contains one or more electrodes for positioning adjacent to the inside or outside wall of a cardiac chamber. The leads have an insulated electrical conductor or conductors for connecting the pulse generator to electrodes in the heart. Such electrode leads typically have lengths of 50 to 70 centimeters.

Although more than one hundred thousand conventional cardiac pacing systems are implanted annually, various well-known difficulties exist, of which a few will be cited. For example, a pulse generator, when located subcutaneously, presents a bulge in the skin that patients can find unsightly, unpleasant, or irritating, and which patients can subconsciously or obsessively manipulate or “twiddle”. Even without persistent manipulation, subcutaneous pulse generators can exhibit erosion, extrusion, infection, and disconnection, insulation damage, or conductor breakage at the wire leads. Although sub-muscular or abdominal placement can address some concerns, such placement involves a more difficult surgical procedure for implantation and adjustment, which can prolong patient recovery.

A conventional pulse generator, whether pectoral or abdominal, has an interface for connection to and disconnection from the electrode leads that carry signals to and from the heart. Usually at least one male connector molding has at least one terminal pin at the proximal end of the electrode lead. The male connector mates with a corresponding female connector molding and terminal block within the connector molding at the pulse generator. Usually a setscrew is threaded in at least one terminal block per electrode lead to secure the connection electrically and mechanically. One or more O-rings usually are also supplied to help maintain electrical isolation between the connector moldings. A setscrew cap or slotted cover is typically included to provide electrical insulation of the setscrew. This briefly described complex connection between connectors and leads provides multiple opportunities for malfunction.

Other problematic aspects of conventional pacemakers relate to the separately implanted pulse generator and the pacing leads. By way of another example, the pacing leads, in particular, can become a site of infection and morbidity. Many of the issues associated with conventional pacemakers are resolved by the development of a self-contained and self-sustainable pacemaker, or so-called leadless pacemaker, as described in the related applications cited above.

Self-contained or leadless pacemakers or other biostimulators are typically fixed to an intracardial implant site by an actively engaging mechanism such as a screw or helical member that screws into the myocardium.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C are embodiments of a leadless cardiac pacemaker or biostimulator.

FIG. 2A is one embodiment of a delivery system for delivering a leadless biostimulator.

FIGS. 2B-2C are close up views of a distal portion of the delivery system of FIG. 2A.

FIGS. 2D-2E are schematic side and cross-sectional views of the delivery system of FIG. 2A.

FIG. 2F is a cutaway view of a handle portion of the delivery system of FIG. 2A.

FIG. 3A is another embodiment of a delivery system for delivering a leadless biostimulator having a tether.

FIGS. 3B-3C are close up views of a distal portion of the delivery system of FIG. 3A.

FIGS. 3D-3E are schematic side and cross-sectional views of the delivery system of FIG. 3A.

FIG. 3F is a cutaway view of a handle portion of the delivery system of FIG. 3A.

FIG. 3G is a close up view of a handle portion of the delivery system of FIG. 3A.

FIG. 3H is one embodiment of a handle portion of a delivery system.

### SUMMARY OF THE DISCLOSURE

In some embodiments, a delivery catheter is provided comprising a handle having a torque knob, a first shaft configured to attach the delivery catheter to a leadless biostimulator, and a second shaft having a proximal portion coupled to the torque knob and a distal portion configured to engage the leadless biostimulator when it is attached to the delivery catheter, the second shaft configured to apply rotational torque to the leadless biostimulator with actuation of the torque knob, wherein the first shaft is coaxially disposed within the second shaft.

In some embodiments, the second shaft further comprises a key configured to mate with a slot on the leadless biostimulator.

In some embodiments, the second shaft further comprises a slot configured to mate with a key on the leadless biostimulator. In one embodiment, the key comprises a shape selected from the group consisting of square, rectangle, triangle, pentagon, hexagon, cross, and “X”.

In some embodiments, the first shaft further comprises a screw configured to engage a threaded hole in the leadless biostimulator. In another embodiment, first shaft further comprises a threaded hole configured to engage a screw on the leadless biostimulator.

In one embodiment, the delivery catheter further comprises a second torque knob coupled to the first shaft, wherein actuation of the second torque knob is configured to rotate the first shaft independently of the second shaft.

A method of delivering a medical device into a patient is also provided, comprising attaching a leadless biostimulator to a delivery catheter, inserting the leadless biostimulator into a patient, advancing the leadless biostimulator to a target tissue, applying torque from a first shaft fully disposed in the delivery catheter to the leadless biostimulator to screw a fixation device of the leadless biostimulator into the target tissue, and unscrewing a second shaft fully disposed in the delivery catheter from the leadless biostimulator to detach the leadless biostimulator from the delivery catheter.

In some embodiments, the applying torque step comprises rotating the first shaft in a first direction. In another embodiment, the unscrewing step comprises rotating the second shaft in a second direction different than the first direction.

In some embodiments, the applying torque step further comprises applying torque from a key disposed on the first shaft of the delivery catheter to a slot disposed on the leadless biostimulator. In another embodiment, the unscrewing step further comprises unscrewing a screw disposed on the second shaft from a threaded hole in the leadless biostimulator.

Another delivery catheter is provided, comprising a shaft configured to apply rotational torque to a leadless biostimulator, lumen disposed within the shaft, the lumen sized and configured to receive a tether of the leadless biostimulator, and a tether lock disposed in the delivery catheter and configured to engage the tether to hold the leadless biostimulator in contact with the delivery catheter.

In some embodiments, the shaft further comprises a key configured to mate with a slot on the leadless biostimulator. In another embodiment, the shaft further comprises a slot configured to mate with a key on the leadless biostimulator. In one embodiment, the key comprises a shape selected from the group consisting of square, rectangle, triangle, pentagon, hexagon, cross, and “X”.

In another embodiment, the tether lock comprises a pin. In some embodiments, he tether lock comprises a button and a locking cam.

A method of delivering a medical device into a patient is provided, comprising applying tension to a tether of a leadless biostimulator to hold the leadless biostimulator in contact with a delivery catheter, inserting the leadless biostimulator into a patient, advancing the leadless biostimulator to a target tissue, applying torque from a shaft of the delivery catheter to the leadless biostimulator to screw a fixation device of the leadless biostimulator into the target tissue, and releasing the tension from the tether to detach the leadless biostimulator from the delivery catheter.

In some embodiments, the applying torque step comprises rotating the shaft. In another embodiment, the applying torque step further comprises applying torque from a key disposed on the shaft of the delivery catheter to a slot disposed on the leadless biostimulator. In yet another embodiment, the applying torque step further comprises applying torque from a slot disposed on the shaft of the delivery catheter to a key disposed on the leadless biostimulator.

### DETAILED DESCRIPTION OF THE INVENTION

Various embodiments for delivering system comprising one or more leadless cardiac pacemakers or biostimulators are described. A leadless cardiac pacemaker can communicate by conducted communication, representing a substantial departure from conventional pacing systems. For example, an illustrative cardiac pacing system can perform cardiac pacing that has many of the advantages of conventional cardiac pacemakers while extending performance, functionality, and operating characteristics with one or more of several improvements.

In some embodiments of a cardiac pacing system, cardiac pacing is provided without a pulse generator located in the pectoral region or abdomen, without an electrode-lead separate from the pulse generator, without a communication coil or antenna, and without an additional requirement on battery power for transmitted communication.

An embodiment of a cardiac pacing system configured to attain these characteristics comprises a leadless cardiac pacemaker that is substantially enclosed in a hermetic housing suitable for placement on or attachment to the inside or outside of a cardiac chamber. The pacemaker can have two or more electrodes located within, on, or near the housing, for delivering pacing pulses to muscle of the cardiac chamber and optionally for sensing electrical activity from the muscle, and for bidirectional communication with at least one other device within or outside the body. The housing can contain a primary battery to provide power for pacing, sensing, and communication, for example bidirectional communication. The housing can optionally contain circuits for sensing cardiac activity from the electrodes. The housing contains circuits for receiving information from at least one other device via the electrodes and contains circuits for generating pacing pulses for delivery via the electrodes. The housing can optionally contain circuits for transmitting information to at least one other device via the electrodes and can optionally contain circuits for monitoring device health. The housing contains circuits for controlling these operations in a predetermined manner.

In some embodiments, a cardiac pacemaker can be adapted for delivery and implantation into tissue in the human body. In a particular embodiment, a leadless cardiac pacemaker can be adapted for implantation adjacent to heart tissue on the inside or outside wall of a cardiac chamber, using two or more electrodes located on or within the housing of the pacemaker, for pacing the cardiac chamber upon receiving a triggering signal from at least one other device within the body.

Self-contained or leadless pacemakers or other biostimulators are typically fixed to an intracardial implant site by an actively engaging mechanism or primary fixation mechanism such as a screw or helical member that screws into the myocardium. Examples of such leadless biostimulators are described in the following publications, the disclosures of which are incorporated by reference: (1) U.S. application Ser. No. 11/549,599, filed on Oct. 13, 2006, entitled “Leadless Cardiac Pacemaker System for Usage in Combination with an Implantable Cardioverter-Defibrillator”, and published as U.S.2007/0088394A1 on Apr. 19, 2007; (2) U.S. application Ser. No. 11/549,581 filed on Oct. 13, 2006, entitled “Leadless Cardiac Pacemaker”, and published as U.S.2007/0088396A1 on Apr. 19, 2007; (3) U.S. application Ser. No. 11/549,591, filed on Oct. 13, 2006, entitled “Leadless Cardiac Pacemaker System with Conductive Communication” and published as U.S.2007/0088397A1 on Apr. 19, 2007; (4) U.S. application Ser. No. 11/549,596 filed on Oct. 13,2006, entitled “Leadless Cardiac Pacemaker Triggered by Conductive Communication” and published as U.S.2007/0088398A1 on Apr. 19, 2007; (5) U.S. application Ser. No. 11/549,603 filed on Oct. 13,2006, entitled “Rate Responsive Leadless Cardiac Pacemaker” and published as U.S.2007/0088400A1 on Apr. 19,2007; (6) U.S. application Ser. No. 11/549,605 filed on Oct. 13, 2006, entitled “Programmer for Biostimulator System” and published as U.S.2007/0088405A1 on Apr. 19, 2007; (7) U.S. application Ser. No. 11/549,574, filed on Oct. 13, 2006, entitled “Delivery System for Implantable Biostimulator” and published as U.S.2007/0088418A1 on Apr. 19, 2007; and (8) International Application No. PCT/U.S.2006/040564, filed on Oct. 13, 2006, entitled “Leadless Cardiac Pacemaker and System” and published as WO07047681A2 on Apr. 26, 2007.

In addition to the primary fixation mechanism, such as a helix, some biostimulators may further include a secondary fixation mechanism to provide another feature for keeping the biostimulator in place within the body. Secondary fixation mechanisms can be either active (e.g., the secondary fixation mechanism can actively engage tissue, either within or outside the heart), or can be passive (e.g., the secondary fixation mechanism is not attached to tissue but rather prevents the biostimulator from moving around in the body in the case of accidental detachment). Further details on secondary fixation mechanisms can be found in U.S. application Ser. No. 12/698,969.

Leadless pacemakers or biostimulators can be delivered to and retrieved from a patient using any of the delivery systems described herein. In some embodiments, a biostimulator is attached or connected to a delivery system and advanced intravenously into the heart. The delivery system can include features to engage the biostimulator to allow fixation of the biostimulator to tissue. For example, in embodiments where the biostimulator includes an active engaging mechanism, such as a screw or helical member, the delivery system can include a docking cap or key configured to engage the biostimulator and apply torque to screw the active engaging mechanism into the tissue. In other embodiments, the delivery system includes clips designed to match the shape of a feature on the biostimulator and apply torque to screw the active engaging mechanism into the tissue.

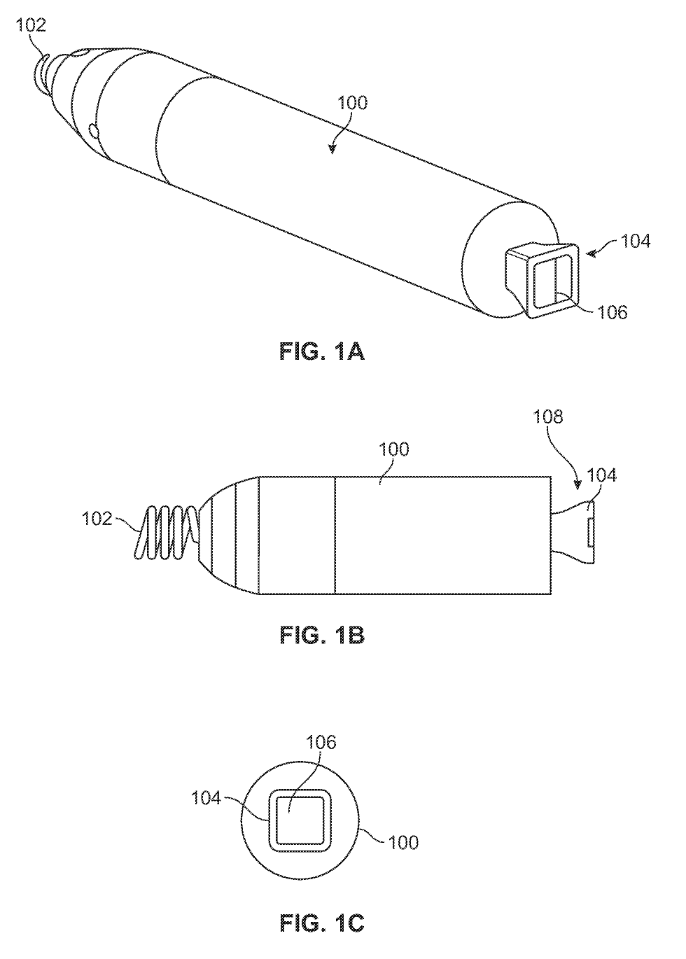

FIGS. 1A-1C show a leadless cardiac pacemaker or leadless biostimulator 100 having an active fixation device or helix 102. The biostimulators can include a hermetic housing at least one electrode disposed thereon. Further details of a typical leadless biostimulator can be found in the U.S. Patent Publications listed above. Additionally, a biostimulator can further include a proximal cap 104 positioned on or near a proximal end of the biostimulator. In FIGS. 1A and 1C, the proximal cap 104 can further comprise an indentation or key 106. The key can be any number of shapes, such as square, triangle, rectangle, hexagon, pentagon, etc, as will be described in further detail below. Referring to FIG. 1B, the proximal cap can further comprise a taper 108 as it is attached to the biostimulator 100. The taper can be grasped or connected to a delivery or extraction catheter, as will be discussed below.

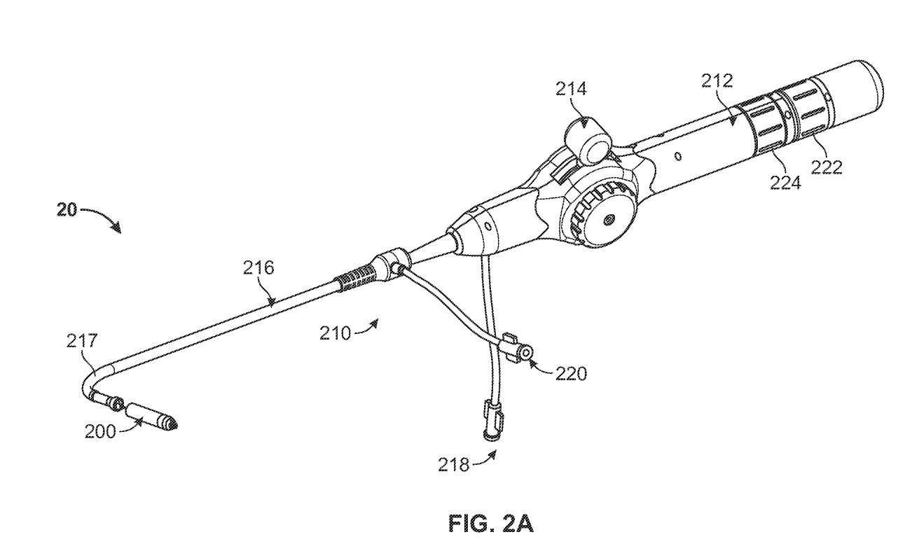

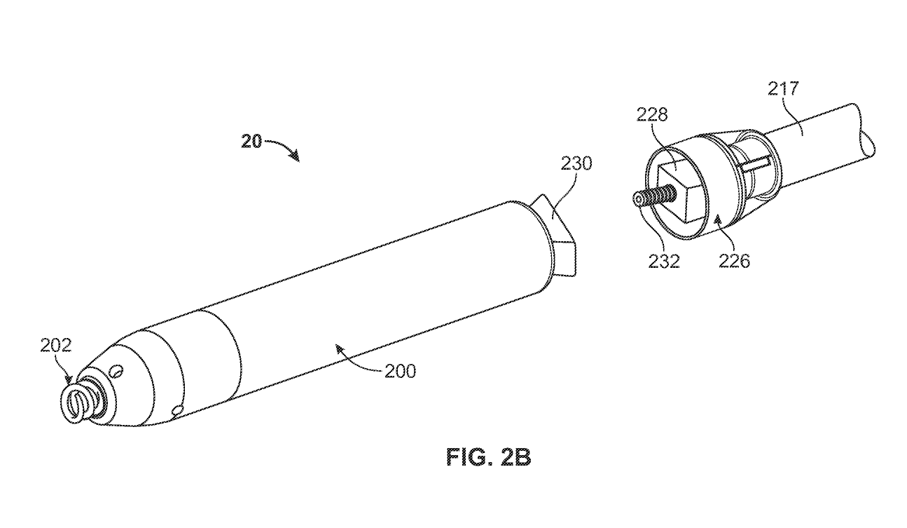

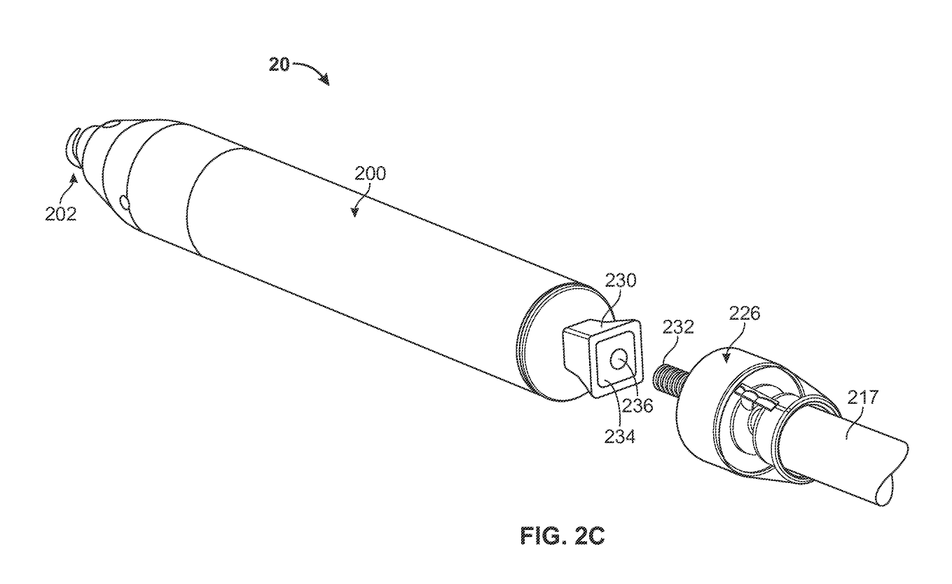

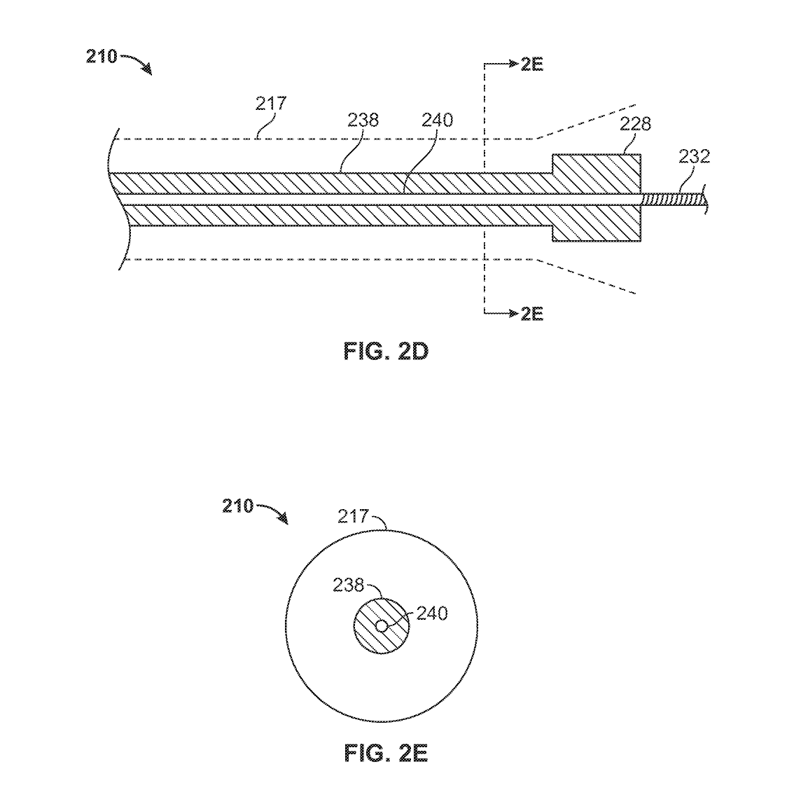

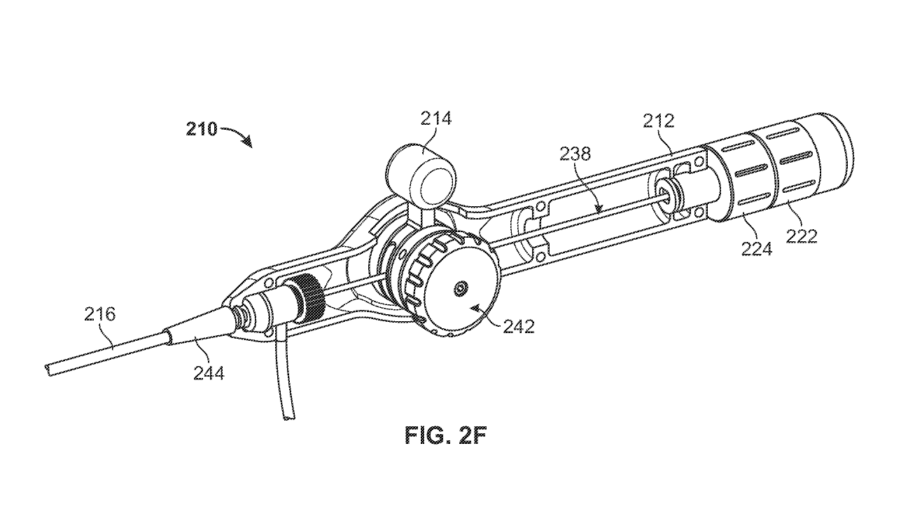

FIG. 2A illustrates a biostimulator delivery system 20 for delivery of a biostimulator 200 into a patient. The delivery system 20 can include delivery catheter 210, handle 212, deflection arm 214, sheath 216, catheter shaft 217, catheter flush port 218, sheath flush port 220, and torque knobs 222 and 224. The deflection arm 214 can be used to steer and guide the catheter during implantation and/or removal of the biostimulator. The catheter flush port 218 and sheath flush port 220 can be used to flush saline or other fluids through the catheter and sheath, respectively. Sheath 216 can be advanced distally over catheter shaft 217 to provide additional steering and support for the delivery catheter during implantation.

FIG. 2B is a close-up view of a distal portion of delivery catheter 210 and biostimulator 200. The biostimulator of FIG. 2B includes a helix 202 for attachment of the biostimulator to tissue. The delivery catheter can include a docking cap 226 having a key 228 sized and configured to mate with a proximal end cap 230 disposed on the biostimulator. The delivery catheter can further include a screw 232 configured to couple with the proximal cap of the biostimulator.

FIG. 2C is another close-up view of the delivery catheter 210 and biostimulator 200 shown in FIG. 2B, but from a different perspective to show the proximal portion of the biostimulator. As shown in FIG. 2C, the proximal end cap 230 includes a cutout or slot 234 sized and configured to mate with the key 228 on catheter 210 (shown in FIG. 2B). Furthermore, the proximal end cap 230 can also include a threaded hole 236 sized and configured to accept and couple to screw 232 of the delivery catheter. In FIGS. 2B-2C, key 228 is shown as a “male” key and slot 234 is shown as a “female” key, but it should be understood that in other embodiments, the “male” key or key 228 can be located on the proximal end cap 230, and the “female” key or slot 234 can be disposed on the delivery catheter. The same can be said for screw 232 and threaded hole 236. It should also be appreciated that key 228 and slot 234 can comprise any number of shapes, such as square, rectangle, triangle, pentagon, hexagon, cross, “X”, etc, so long as key 228 fits within and can apply rotational torque to slot 234.

FIG. 2D is a schematic diagram of delivery catheter 210, showing coaxial shafts 238 and 240 coupled to key 228 and screw 232, respectively, and extending through the length of the catheter shaft 217. Since shafts 238 and 240 are arranged in a coaxial configuration within catheter shaft 217, the key 228 and screw 232 can be rotated independently from one another during implantation and/or removal of the biostimulator into tissue. FIG. 2E is a cross sectional view of FIG. 2D along line 2E-2E, showing the relative positions and sizes of shafts 238 and 240 within the delivery catheter 210.

FIG. 2F is a cutaway view of handle 212 of delivery catheter 210, showing how coaxial shafts 238 and 240 (not shown because it is disposed within shaft 238) are connected to the handle. As shown in FIG. 2F, shaft 238 runs from the distal end of the catheter shaft (from key 228 in FIG. 2B) and terminates at torque knob 224 in handle 212. Similarly, shaft 240 (not shown in FIG. 2F because it is disposed coaxially within shaft 238) runs from the distal end of the catheter shaft (from screw 232 in FIG. 2B) and terminates at torque knob 222 in handle 212. Rotation of torque knob 224 by a user, such as a physician, will cause rotation of shaft 238. Similarly, rotation of torque knob 222 will cause rotation of shaft 240. In addition, FIG. 2F shows deflection arm lock 242 and catheter strain relief 244. Deflection arm lock 242 can be used to lock the distal portion of the delivery catheter in place when the catheter has been bent or steered using deflection arm 214. Catheter strain relief 242 provides a smooth transition between the handle and the catheter shaft so as to prevent kinking at the junction between the shaft and handle.

Referring to FIGS. 2A-2F, it can now be understood how biostimulator 200 can be delivered and attached to tissue, and then released from delivery system 20. In FIGS. 2B-2C, docking cap 226 of delivery catheter 210 can be advanced over proximal end cap 230 of biostimulator 200 so that key 228 fits within and is mated to slot 234. Screw 232 can then be inserted into hole 236 and attached to biostimulator 200 by rotating torque knob 222 (and thus rotating shaft 240 and screw 232) so as advance and engage the screw 232 into threaded hole 236. When torque knob 222 is rotated to screw the delivery catheter into the biostimulator, torque knob 224 can be left alone (e.g., not rotated) so that key 228 engages and applies torque to slot 234.

Next, the biostimulator and delivery system can be inserted into the patient and advanced to the target location (e.g., the biostimulator can be advanced into the cardiac chamber) as known in the art.

Upon reaching the target tissue, both torque knobs 222 and 224 can be rotated together to cause helix 202 of biostimulator 200 to engage and become inserted into tissue. By rotating the torque knobs together, both coaxial shafts 238 and 240 (and thus key 228 and screw 232) are rotated together, causing the biostimulator and helix 202 to rotate or screw into tissue. Once the helix is fully inserted into tissue, torque knob 224 can be held in place, and torque knob 222 can be unscrewed, causing screw 232 to disengage from threaded hole 236 of biostimulator 200. Once the delivery catheter 210 is disengaged from the biostimulator, the catheter can be removed from the patient, leaving the biostimulator in place at the target tissue.

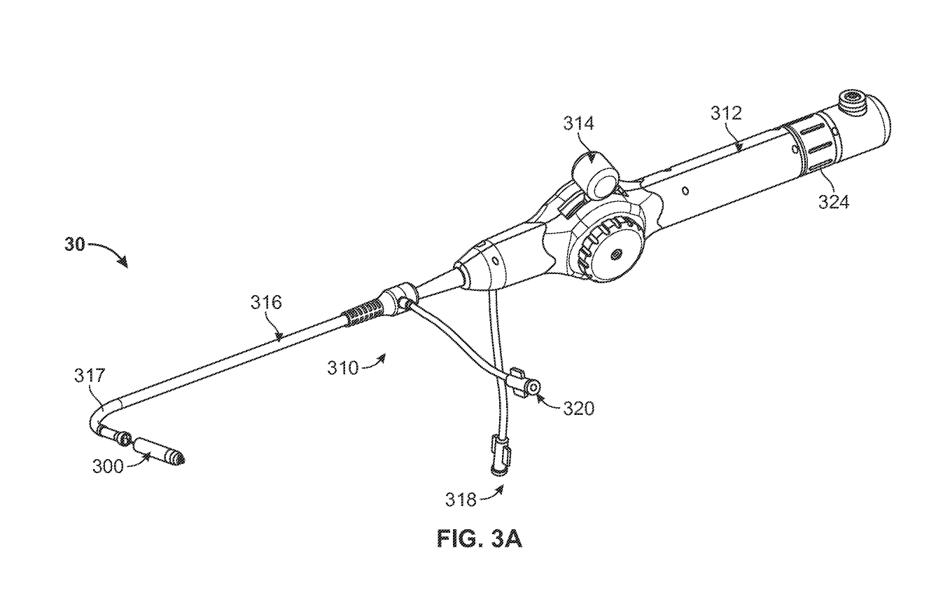

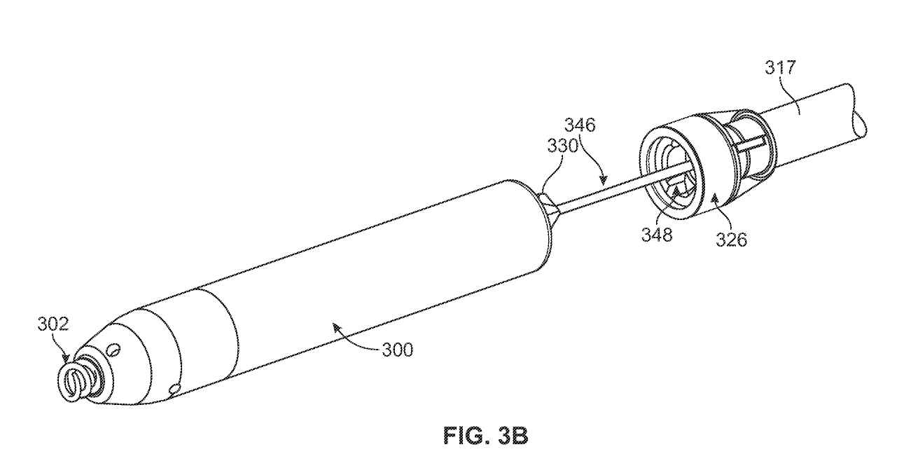

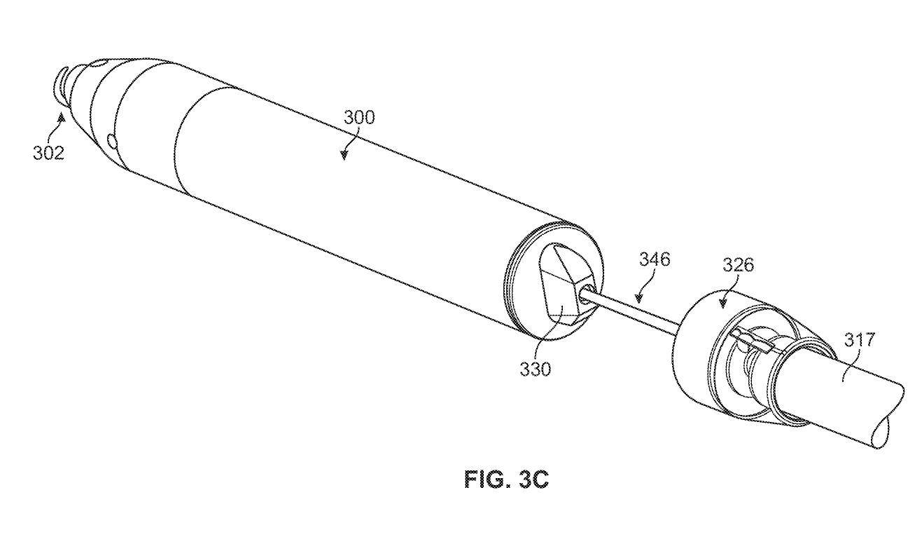

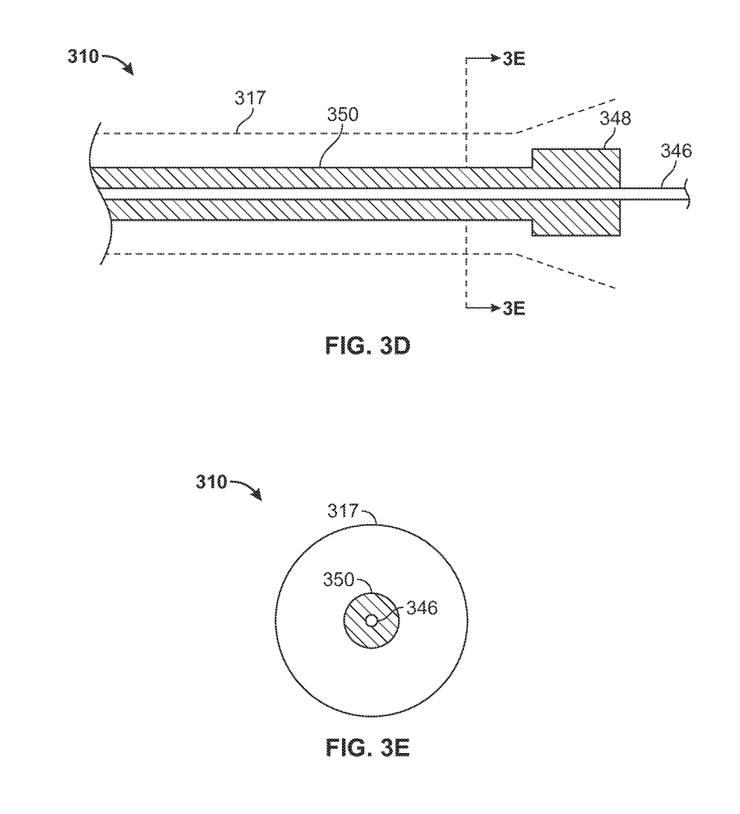

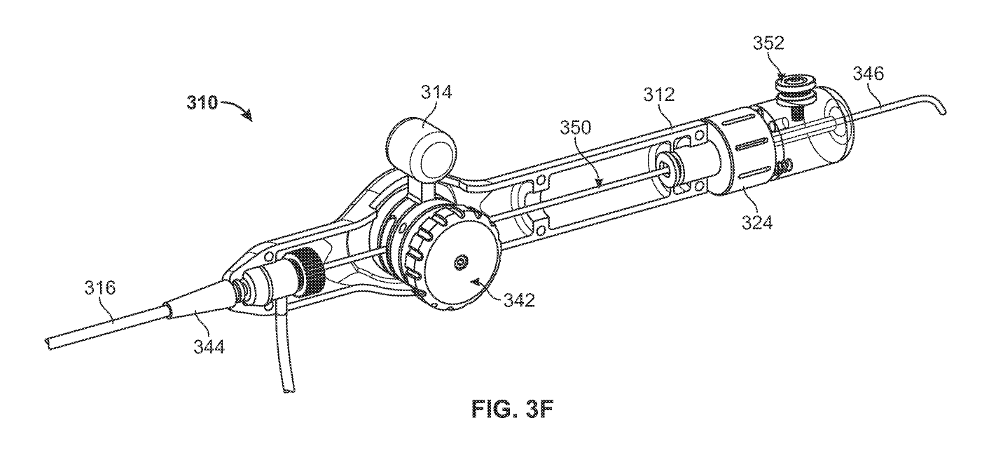

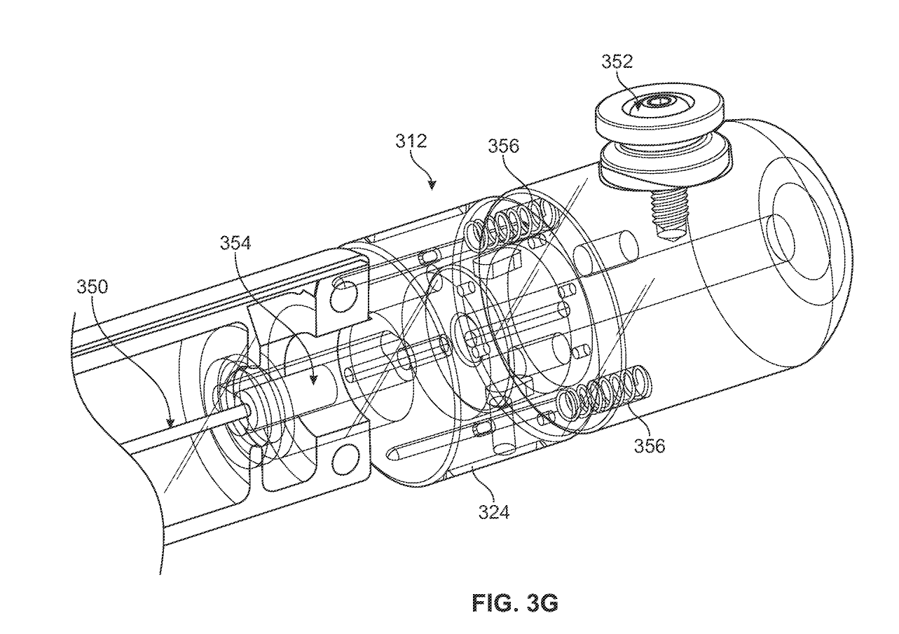

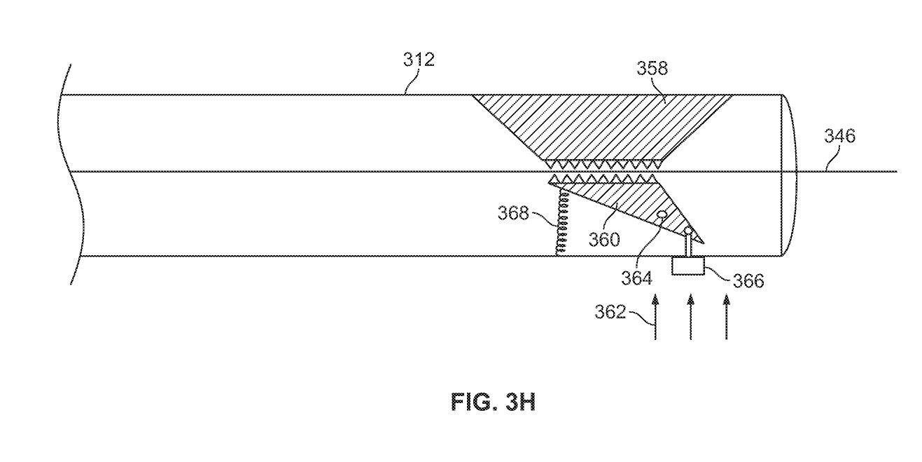

FIG. 3A illustrates a biostimulator delivery system 30 for delivery of a biostimulator 300 into a patient. The delivery system 30 can correspond to delivery system 20 of FIGS. 2A-2F, so delivery catheter 310, handle 312, deflection arm 314, sheath 316, catheter shaft 317, catheter flush port 318, sheath flush port 320, and torque knob 324 of FIG. 3A can correspond, respectively, to delivery catheter 210, handle 212, deflection arm 214, sheath 216, catheter shaft 217, catheter flush port 218, sheath flush port 220, and torque knob 224 of FIG. 2A.

FIG. 3B is a close-up view of a distal portion of delivery catheter 310 and biostimulator 300. The biostimulator of FIG. 3B includes a helix 302 for attachment of the biostimulator to tissue as well as a secondary fixation mechanism or tether 346. In some embodiments, the tether may be either a conductive or non-conductive material. As described above, a secondary fixation mechanism or tether can be used to provide a second point of attachment between the biostimulator and the patient. The delivery catheter can include a docking cap 326 having a slot 348 sized and configured to mate with a proximal end cap 330 disposed on the biostimulator.

FIG. 3C is another close-up view of the delivery catheter 310 and biostimulator 300 shown in FIG. 3B, but from a different perspective to show the proximal portion of the biostimulator. As shown in FIG. 3C, the proximal end cap 330 can be sized, shaped, and configured to mate with the slot 348 on catheter 310 (shown in FIG. 3B). Furthermore, the proximal end cap 330 can also be the point of attachment of tether 346 to the biostimulator 300. In FIGS. 3B-3C, proximal end cap 330 is shown as a “male” key and slot 348 is shown as a “female” key, but it should be understood that in other embodiments, the “male” key can be located on the delivery catheter, and the “female” key can be disposed on or in the proximal end cap. It should also be appreciated that proximal end cap 330 and slot 348 can comprise any number of shapes, such as square, rectangle, triangle, pentagon, hexagon, etc, so long as slot 348 fits around and can apply torque to proximal end cap 330.

FIG. 3D is a schematic diagram of delivery catheter 310, showing shaft 350 and tether 346 disposed within a lumen inside shaft 350. Since shaft 350 and tether 346 are arranged in a coaxial configuration within catheter shaft 317, the shaft 350 and slot 348 can be rotated independently from catheter shaft 317 and tether 346 during implantation and/or removal of the biostimulator into tissue. FIG. 3E is a cross sectional view of FIG. 3D along line 3E-3E, showing the relative positions and sizes of shaft 350 and tether 346 within the delivery catheter 310.

FIG. 3F is a cutaway view of handle 312 of delivery catheter 310, showing how shaft 350 and tether 346 are connected to the handle 312. As shown in FIG. 3F, shaft 350 runs from the distal end of the catheter shaft (from key slot 348 in FIG. 3B) and terminates at torque knob 324. Tether 346 runs from the biostimulator through the torque shaft 350 and extends beyond the proximal end of handle 312. Tether 346 is moveable within the catheter and the handle. In one embodiment, tether lock 352 can comprise a pin and tether 346 can extend beyond the handle and wrap around tether lock 352 to hold the tether taut and in place during implantation and/or removal of the biostimulator. Rotation of torque knob 324 by a user, such as a physician, will cause rotation of shaft 350 and slot 348.

Referring to FIGS. 3A-3F, it can now be understood how biostimulator 300 can be delivered and attached to tissue, and then released from delivery system 30. In FIGS. 3B-3C, docking cap 326 and slot 348 of delivery catheter 310 can be advanced over proximal end cap 330 of biostimulator 200 so that slot 348 fits over and is mated to end cap 330. Tension can then be applied to tether 346 to pull and hold biostimulator 300 tight against and in contact with delivery catheter 310. Referring to FIG. 3F, the tether can be wound around tether lock 352 to hold tether 346, and thus biostimulator 300, in place on the delivery catheter.

Next, the biostimulator and delivery system can be inserted into the patient and advanced to the target location (e.g., the biostimulator can be advanced into the cardiac chamber), as known in the art.

Upon reaching the target tissue, torque knob 324 can be rotated to cause helix 302 of biostimulator 300 to engage and become inserted into tissue. By rotating the torque knob, shaft 350 causes slot 348 to engage and apply torque to proximal end cap 330, forcing the helix to rotate and screw into tissue. Once the biostimulator is fully inserted into tissue, the tether can be removed from tether lock 352, releasing the tension in tether 346 and causing biostimulator to be free to pull away from delivery catheter 310. The delivery catheter 310 is free to be disengaged from the biostimulator, and the catheter can be removed from the patient over the tether, leaving the biostimulator in place at the target tissue. The tether 346 can then be attached to the desired tissue to provide a secondary anchor for the biostimulator.

FIG. 3G is a close-up view of a proximal portion of handle 312, including torque knob 324, shaft 350, tether lock 352, torque puck 354, and tether tension springs 356. During steering of the delivery catheter, the catheter foreshortens as it is deflected. The length of torque shaft 350 does not change since it is typically made of stainless steel tube and coil. To compensate for the length change between the catheter and the torque shaft during deflection, the distal tip of the torque shaft is fixed at the distal tip of the catheter and the proximal end of the torque shaft is allowed to float inside the handle. Torque puck 354 is attached to the proximal end of the torque shaft 350 to allow the torque shaft to move longitudinally during catheter deflection. The torque puck can include a keyed shape that allows it to move longitudinally but remain able to be rotated radially so as to turn the torque shaft (and thus the key at the distal end of the torque shaft). The tether tension springs can be attached to the tether to keep the tether under tension (and thereby keep the LCP docked to the tip of the delivery catheter) during delivery and navigation of the delivery catheter.

FIG. 3H is a close-up view of an alternative embodiment of a proximal portion of handle 312, in which the tether lock 352 of FIG. 3G has been replaced with surface 358 and locking cam 360. Instead of wrapping the tether around tether lock 352, as described above, in the embodiment of FIG. 3H the tether 346 can be frictionally held in place between surface 358 and cam 360. The locking cam can include a spring 368 and hinge 364 to cause locking cam 360 to apply a return force against the surface 358 and thus hold the tether in place. Pushing button 366 inwards towards the handle, as indicated by arrows 362, can cause the locking cam to pivot on hinge 364 to release the tether.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis, for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A delivery catheter, comprising:
a handle having a torque knob;
a first shaft configured to attach the delivery catheter to a leadless biostimulator; and
a second shaft having a proximal portion coupled to the torque knob and a distal portion configured to engage the leadless biostimulator when it is attached to the delivery catheter, the second shaft configured to apply rotational torque to the leadless biostimulator with actuation of the torque knob, wherein the first shaft is coaxially disposed within the second shaft.

2. The delivery catheter of claim 1 wherein the second shaft further comprises a key configured to mate with a slot on the leadless biostimulator.

3. The delivery catheter of claim 1 wherein the second shaft further comprises a slot configured to mate with a key on the leadless biostimulator.

4. The delivery catheter of claim 2 wherein the key comprises a shape selected from the group consisting of square, rectangle, triangle, pentagon, hexagon, cross, and “X”.

5. The delivery catheter of claim 1 wherein the first shaft further comprises a screw configured to engage a threaded hole in the leadless biostimulator.

6. The delivery catheter of claim 1 first shaft further comprises a threaded hole configured to engage a screw on the leadless biostimulator.

7. The delivery catheter of claim 1 further comprising a second torque knob coupled to the first shaft, wherein actuation of the second torque knob is configured to rotate the first shaft independently of the second shaft.

8. A method of delivering a medical device into a patient, comprising:
attaching a leadless biostimulator to a delivery catheter;
inserting the leadless biostimulator into a patient;
advancing the leadless biostimulator to a target tissue;
applying torque from a first shaft fully disposed in the delivery catheter to the leadless biostimulator to screw a fixation device of the leadless biostimulator into the target tissue; and
unscrewing a second shaft fully disposed in the delivery catheter from the leadless biostimulator to detach the leadless biostimulator from the delivery catheter.

9. The method of claim 8 wherein the applying torque step comprises rotating the first shaft in a first direction.

10. The method of claim 9 wherein the unscrewing step comprises rotating the second shaft in a second direction different than the first direction.

11. The method of claim 8 wherein the applying torque step further comprises applying torque from a key disposed on the first shaft of the delivery catheter to a slot disposed on the leadless biostimulator.

12. The method of claim 8 wherein the unscrewing step further comprises unscrewing a screw disposed on the second shaft from a threaded hole in the leadless biostimulator.

13. A delivery catheter, comprising:
a shaft configured to apply rotational torque to a leadless biostimulator;
a lumen disposed within the shaft, the lumen sized and configured to receive a tether of the leadless biostimulator; and
a tether lock disposed in the delivery catheter and configured to engage the tether to hold the leadless biostimulator in contact with the delivery catheter.

14. The delivery catheter of claim 13 wherein the shaft further comprises a key configured to mate with a slot on the leadless biostimulator.

15. The delivery catheter of claim 13 wherein the shaft further comprises a slot configured to mate with a key on the leadless biostimulator.

16. The delivery catheter of claim 15 wherein the key comprises a shape selected from the group consisting of square, rectangle, triangle, pentagon, hexagon, cross, and “X”.

17. The delivery catheter of claim 13 wherein the tether lock comprises a pin.

18. The delivery catheter of claim 13 wherein the tether lock comprises a button and a locking cam.

19. A method of delivering a medical device into a patient, comprising:
applying tension to a tether of a leadless biostimulator to hold the leadless biostimulator in contact with a delivery catheter;
inserting the leadless biostimulator into a patient;
advancing the leadless biostimulator to a target tissue;
applying torque from a shaft of the delivery catheter to the leadless biostimulator to screw a fixation device of the leadless biostimulator into the target tissue; and
releasing the tension from the tether to detach the leadless biostimulator from the delivery catheter.

20. The method of claim 19 wherein the applying torque step comprises rotating the shaft.

21. The method of claim 19 wherein the applying torque step further comprises applying torque from a key disposed on the shaft of the delivery catheter to a slot disposed on the leadless biostimulator.

22. The method of claim 19 wherein the applying torque step further comprises applying torque from a slot disposed on the shaft of the delivery catheter to a key disposed on the leadless biostimulator.
